(19) United States
(12) Patent Application Publication
(10) Pub. No.: US 2009/0209449 A1
(43) Pub. Date: Aug. 20, 2009

(54) NOVEL WOODY ODORANTS

(76) Inventors: Muhammed Majeed, Psicataway, NJ (US); Parthasarathy Rajasekharan, Bangalore (IN); Chandramohan Thiruvanath Variam, Bangalore (IN); Mahesh Rajamony, Bangalore (IN)

Correspondence Address: SABINSA CORPORATION, 70 ETHEL ROAD WEST, UNIT 6, PISCATAWAY, NJ, 08854, US

(21) Appl. No.: 12/034,251

(22) Filed: Feb. 20, 2008

(51) Int. Cl. A61K 8/33; C07C 35/17; A61Q 13/00

(52) U.S. Cl. 512/22; 568/828

(57) ABSTRACT

The present invention relates to novel substituted cyclohexyl monohydric alcohols possessing woody odor which are useful as fragrance materials. The invention also provides method for synthesis of the above compounds. The invention also provides fragrance compositions which utilize the compounds of the invention to impart woody aroma to such compositions.

The compounds of the invention have the general formula as under

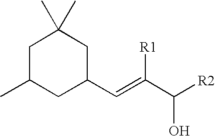

### BACKGROUND OF THE INVENTION

There has always been a need for new and novel synthetic chemicals having desirable fragrance properties. Such chemicals are used either to replace expensive natural fragrance materials or to provide much needed value addition to existing olfactory properties.

Certain monocyclic chemicals possessing sandalwood odor have been prepared in the laboratory. A chemical (U.S. Pat. No.4,046,716 issued in 1977) having the structure 1 has been reported to have a mild sandalwood odor.

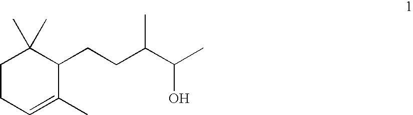

The monocyclic alcohols 2 (U.S. Pat. No. 4,052,341 issued 1976) and (U.S. Pat. No. 4,610,813 issued in 1986) also possess sandalwood odor.

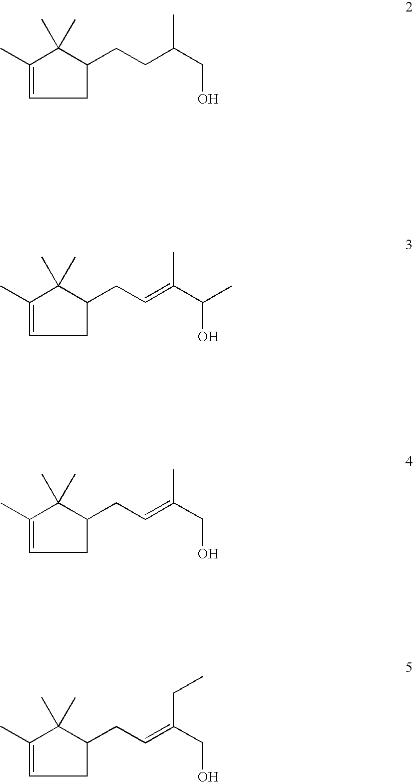

The classical sandalwood rules, and results of more recent studies underline the importance of the distance between the hydroxyl group and a bulky moiety, that preferably contains an electron rich fragment, i.e. a double bond, an ether functionalitiy or a cyclopropane ring The often flexible spacer linking these fragments should be branched next to the hydroxyl bearing carbon. The necessary structural features of the bulky part of a sandalwood smelling substance are also difficult to define [G. Frater et al./Tetrahedron 54(1998 ) page 7639]

A thorough study of sandalwood type compounds revealed that one of the efficient synthon for the preparation of sandalwood odorant compounds is constituted by 6.

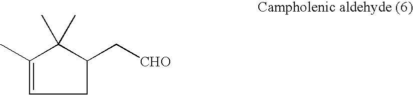

The above study was useful in identifying the necessary structural features required by a given chemical to develop the desired sandalwood odor.

In a similar manner, for the present invention 3,5,5-Trimethyl cyclohexane carboxaldehyde (7) was selected as the synthon for preparation of the cyclic monohydric alcohols.

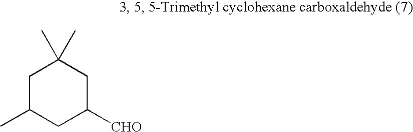

### FIELD OF INVENTION

The present invention relates to novel cyclohexyl monohydric alcohols as odorants, particularly those possessing a fine woody aroma, useful as fragrances suitable for incorporation in fine fragrances, cosmetics, toiletries and related applications. The invention also provides an economical and commercially viable process from readily available and low cost raw materials for preparing these compounds.

### SUMMARY OF THE INVENTION

The present invention is directed to the discovery of novel compounds and the use of these compounds as fragrance chemicals, separately or as a mixture, as perfume ingredients for the preparation of perfumes, colognes, personal care products etc.

In a first embodiment of the invention we have discovered the following novel compounds of the formula I,II and III set forth below:

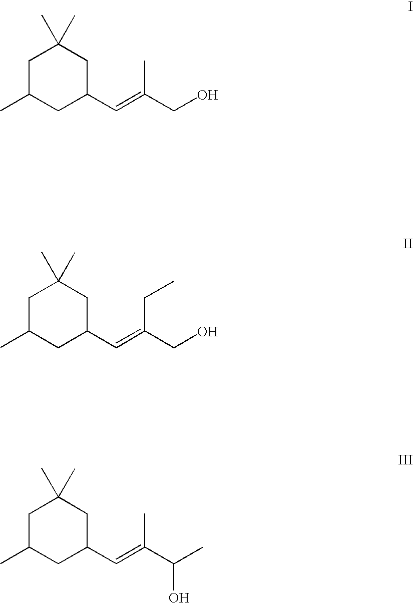

These novel alcohols I,II and III are prepared by reacting the aldehyde of the formula

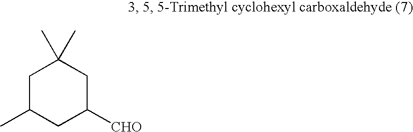

with Propionaldehyde, Butyraldehyde and 2-Butanone in presence of a base followed by Sodium borohydride reduction to get the compounds I,II and III respectively. It has been found that these novel cyclohexyl monohydric alcohols of the present invention possess fragrant woody aroma. They exhibit spicy nuances rendering them as useful materials for oriental-type fragrance applications. The amounts of these novel alcohols effective to impart fragrance is about 0.1% to about 3% by weight in conventional fragrance ingredients.

### DETAILED DESCRIPTION OF THE INVENTION

The preferred alcohols of the present invention are:

2-Methyl-3(3,5,5-trimethyl cyclo hexane)-2-propenol (I)

2-Ethyl-3(3,5,5-trimethyl cyclo hexane)-2-propenol (II)

2-Methyl-4(3,5,5-trimethyl cyclo hexane)-3-propen-2-ol (III)

The following reaction schemes illustrate the process of the present invention for conveniently and efficiently preparing novel alcohols I, II, and III, which are useful individually or as mixtures in fragrance applications.

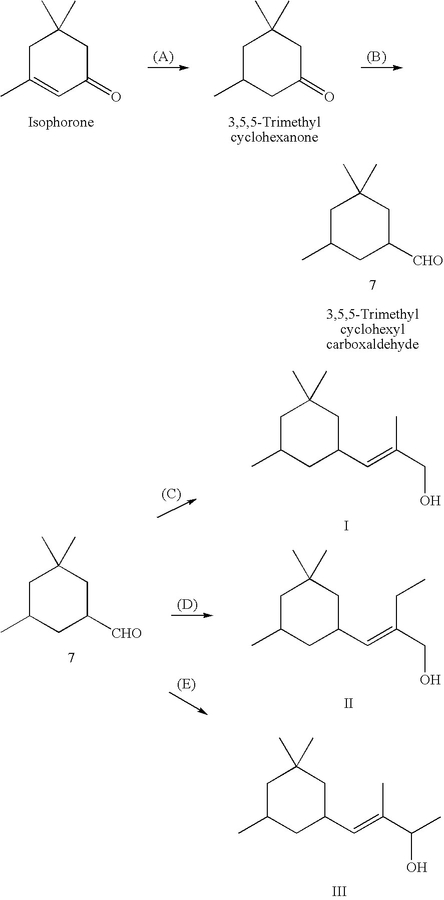

Hydrogenation of Isophorone was carried out using a suitable catalyst (Pd Charcoal) to get the saturated product, Dihydro isophorone. This ketone was condensed with a holoacetic ester in the presence of metal alkoxide to give a glycidic ester, which is then converted into the free acid by careful hydrolysis and decarboxylation.

It has been found that this compounds I, II and III have advantageous olfactory properties and can be used as perfume ingredients.

In a second embodiment, the present invention is a method for enhancing a perfume by incorporating an olfactory acceptable amount of compounds I, II and III. The above materials can also be incorporated into fragrance formulations to provide unique fragrances.

### Illustration of Preferred Embodiments

The following examples serve to illustrate embodiments of the present invention. It will be understood that these examples are illustrative and the invention is to be restricted thereto only as indicated in the appended claims. As used herein all percentages are weight percent. DEP is understood to mean diethylphthalate. PEA is understood to mean Phenyl ethyl alcohol and DHM is understood to mean Dihydromyrcenol.

Synthesis:

This subsection shows by example and illustration how the compounds of this invention can be prepared.

### EXAMPLE 1

### Preparation of 3,5,5-trimethyl cyclohexanone

5% Palladium on carbon (10 grams) is added to the mixture of 100 grams of Isophorone (3,5,5-trimethyl-2-cyclohexenone) and 500 mls methanol. The mixture is hydrogenated at 25° C. to 30° C. under 5 kg hydrogen pressures for 2 hours. The palladium on carbon is filtered and then removed methanol by distillation at reduced pressure to give 100 grams of 3,5,5-trimethyl cyclohexanone. Spectral data were consistent with the structure given.

### EXAMPLE 2

### Preparation of 3,5,5-Trimethyl cyclohexane carboxaldehyde

Sodium methoxide (38 grams) is added portion wise over a 1-hour period to a stirred mixture of 100 grams of 3,5,5-trimethyl cyclohexenone and 80 grams of methyl chloroacetate at −5° C. to 0° C. The resulting thick mass is stirred for an additional 4 hours at −5° C. to 0° C. A solution of 42 grams of sodium hydroxide in 266 mls of methanol is then added slowly at 0° C. and the resulting solution is stirred at room temperature for 3 hours.

The reaction mass is then poured into 500 mls of water with stirring. The aqueous reaction mass is extracted twice with 100 mls of toluene. The toluene layer is discarded and the aqueous layer is acidified with 70 mls of conc-HCl (pH˜2). The aqueous layer is extracted with three 150 mls of toluene. The combined toluene layers are washed with 250 mls of water. The toluene layer is removed by distillation at reduced pressure. Distillation under vacuum accompanied by release of carbon dioxide affords fractions containing 50 grams of 3,5,5-trimethyl cyclohexane carboxaldehyde. Spectral data were consistent with the structure given.

### EXAMPLE 3

### Preparation of 2-Methyl-3-(3,5,5-trimethyl cyclohexyl)-2-propenal

The mixture of 190 grams of 3,5,5-trimethyl cyclohexane carboxaldehyde and 128.8 grams of propionaldehyde is added to the methoxide solution which is made by dissolving 2.8 of grams sodium in 500 mls of methanol at 55 to 60° C. The resulting solution is stirred at the same temperature for 1 hour. The reaction mass is quenched into 1000 mls of water with stirring and then neutralized by 7 grams of acetic acid. The aqueous layer is extracted with three 150 mls of Hexane. The combined Hexane layers are washed with 250 mls of water. The Hexane layer is removed by distillation at reduced pressure. Distillation under vacuum give the fraction containing 75 grams of 2-Methyl-3-(3,5,5-trimethyl cyclohexyl)-2-propenal.

### EXAMPLE 4

### Preparation of 2-Methyl-3-(3,5,5-trimethyl cyclohexane)-2-propenol (I)

A solution of 5.7 grams of sodium borohydride and of 0.5 grams of sodium hydroxide in 20 mls of water is then added slowly to the mixture of 75 grams of 2-Methyl-3-(3,5,5-trimethyl cyclo hexane)-2-propenal and 75 mls of methanol at 15 to 20° C. The resulting solution is stirred at room temperature for 3 hours. The reaction mass is quenched into 200 mls of water with stirring and then neutralized by Acetic acid. The aqueous layer is extracted with three 150 mls of Hexane. The combined Hexane layers are washed with 250 mls of water. The Hexane layer is removed by distillation at reduced pressure. Distillation under vacuum give the fraction containing 60 grams of 2-Methyl-3(3,5,5-trimethyl cyclohexane)-2-propenol.

BP:88° C./1 mm; IR:3340 cm-1; NMR:0.8-0.9 ppm (m,12H), 1.3 ppm(m,2H),1.6(m,2H)1.67 ppm(d,3H), 2.4 ppm(m,1H),3.96 ppm(s,2H), 5.15 ppm(d,1H) MS:M+196;

### EXAMPLE 5

### Preparation of 2-Ethyl-3-(3,5,5-trimethyl cyclohexane)-2-propenal

The mixture of 200 grams of 3,5,5-trimethyl cyclo hexane carboxaldehyde and 168.3 grams of Butyraldehyde is added to the methoxide solution which is made by dissolving 15 of grams sodium in 200 mls of methanol at 55° C. to 60° C. The resulting solution is stirred at the same temperature for 1 hour. The reaction mass is quenched into 400 mls of water with stirring and then neutralized by 30 grams of acetic acid. The aqueous layer is extracted with three 150 mls of Hexane. The combined Hexane layers are washed with 250 mls of water. The Hexane layer is removed by distillation under reduced pressure. Distillation under vacuum give the fraction containing 100 grams of 2-Ethyl-3(3,5,5-trimethyl cyclo hexane)-2-propenal.

### EXAMPLE 6

### Preparation of 2-Ethyl-3(3,5,5-trimethyl cyclohexane)-2-propenol (II)

A solution of 7.1 grams of sodium borohydride and of 0.5 grams of sodium hydroxide in 40 mls of water is then added slowly to the mixture of 100 grams of 2-Ethyl-3(3,5,5-trimethyl cyclo hexane)-2-propenal and 100 mls of methanol at 15 to 20° C. The resulting solution is stirred at room temperature for 3 hours. The reaction mass is quenched into 200 mls of water with stirring and then neutralized by Acetic acid. The aqueous layer is extracted with three 150 mls of Hexane. The combined Hexane layers are washed with 250 mls of water. The Hexane layer is removed by distillation at reduced pressure. Distillation under vacuum give the fraction containing 75 grams of 2-Ethyl-3(3,5,5-trimethyl cyclo hexane)-2-propenol.

BP:96° C./1 mm; IR:3350, 2957,2913,1450 cm−1 NMR:0.5 0.9 ppm(m,11H), 1.01 ppm(t,3H),1.3(m,2H)1.58 ppm(m,2H),2.12(q,2H), 2.4 ppm(m,1H) 4.01 ppm(d,2H), 5.13 ppm(d,1H) MS: M+210,

### EXAMPLE 7

### Preparation of 2-Methyl-4-(3,5,5-trimethyl cyclo hexane)-3-buten-2-one

Methyl ethyl ketone (702 grams) is added to a solution of 45.5 grams of potassium hydroxide in 750 mls of methanol at 30° C. and then added 250 grams of 3,5,5-trimethyl cyclo hexane carboxaldehyde at 30° C. The resulting solution is stirred at 30° C. for 3 hours. The reaction mass is quenched into 1500 mls of water with stirring and then neutralized by Acetic acid. The aqueous layer is extracted with three 250 mls of Hexane. The combined Hexane layers are washed with 250 mls of water. The Hexane layer is removed by distillation at reduced pressure. Distillation under vacuum give the fraction containing 175 grams of 2-Methyl-4(3,5,5-trimethyl cyclo hexane)-2-propen-2-one.

### EXAMPLE 8

### Preparation of 2-Methyl-4-(3,5,5-trimethyl cyclohexyl)-3-buten-2-ol (III):

A solution of 15.6 grams of sodium borohydride and of 0.5 grams of sodium hydroxide in 100 mls of water is then added slowly to the mixture of 175 grams of 2-Methyl-3(3,5,5-trimethyl cyclo hexane)-3-propen-2-one and 875 mls of methanol at 15 to 20° C. The resulting solution is stirred at room temperature for 3 hours. The reaction mass is quenched into 2000 mls of water with stirring and then neutralized by Acetic acid. The aqueous layer is extracted with three 500 mls of Hexane. The combined Hexane layers are washed with 500 mls of water. The Hexane layer is removed by distillation at reduced pressure. Distillation under vacuum give the fraction containing 125 grams of 2-Methyl-4(3,5,5-trimethyl cyclo hexane)-3-propen-2-ol.

BP:98°/1 mm, IR:3315,2951,2915,1451 cm−, 1 NMR: 0.6, 1.0 ppm(m,14H),1.2 ppm (d,3H),1.63 ppm(d,6H),(2.4 ppm(m,1 H) 4.16 ppm(q,1 H), 5.147 ppm(d,1 H) MS:M+210

The following fragrances were prepared using compounds I,II and III of the present invention:

### EXAMPLE 9

### Incorporation of 2-Methyl-3-(3,5,5-trimethyl cyclohexyl)-2-propenol (Compound I)of the Present Invention

| Ingredients | weight(g) |
|---|---|
| Geraniol | 500 |
| Citronellol | 300 |
| PEA | 100 |
| Rhodinol | 60 |
| Petitgrain | 25 |
| Diphenyl oxide | 50 |
| Linalool | 50 |
| Alpha lonone | 50 |
| Citronellyl Tiglate | 25 |
| Acetyl Isoeugenol | 25 |
| Alpha Damascene | 5 |
| Beta Damascone | 5 |
| Naturanol | 25 |
| Compound I | 25 |
| DEP | 250 |
| | 1500 |

The above fragrance was found to be a pleasing fragrance with woody and rose notes.

### EXAMPLE 10

### Incorporation of 2-Ethyl-3-(3,5,5-trimethyl cyclohexane)-2-propenol (compound II) of the Present Invention

| Ingredients | weight(g) |
|---|---|
| Bergamot oil | 200 |
| Lemon oil | 100 |
| Orange oil | 50 |
| Rosemary oil | 5 |
| Comound II | 20 |
| Galaxolide | 15 |
| Sandanol | 25 |
| Vanillin | 5 |
| Petitgrain | 35 |
| Greenarome | 3 |
| DEP | 142 |
| | 600 |

The above fragrance was found to be a pleasing fragrance with woody and spicy notes.

### EXAMPLE 11

### Incorporation of 2-Methyl-4-(3,5,5-trimethyl cyclo hexane)-3-buten-2-ol (Compound III) of the Present Invention

| Ingredients | weight(g) |
|---|---|
| Isomohanol | 110 |
| Compound III | 50 |
| Supersantol | 25 |
| Pent-3-en-2-one | 61 |
| Galaxolide | 78 |
| Cumin oil | 0.5 |
| Vanillin | 51 |
| Ambrester | 37 |
| Petitgrain oil | 35 |
| PEA | 53 |
| Bergamot oil | 32 |
| Cedarwood oil | 47 |
| Methyl ionone | 56 |
| Geranium oil RC | 36 |
| Lyral | 33 |
| Benzyl salicylate | 10 |
| Lavender oil | 10 |
| DHM | 20 |
| Linalool | 20 |
| DEP | 209.5 |
| | 1000 |

The above fragrance was found to be a pleasing fragrance with woody and sandal notes.

All the above fragrance formulations were presented to demonstrate the effectiveness of the compounds of the present invention in enhancing, improving or modifying the performance of the formulations in which they are incorporated.

What is claimed is:

1. A method for improving, enhancing or modifying a fragrance through the addition of an olfactorily acceptable amount of the following compounds:

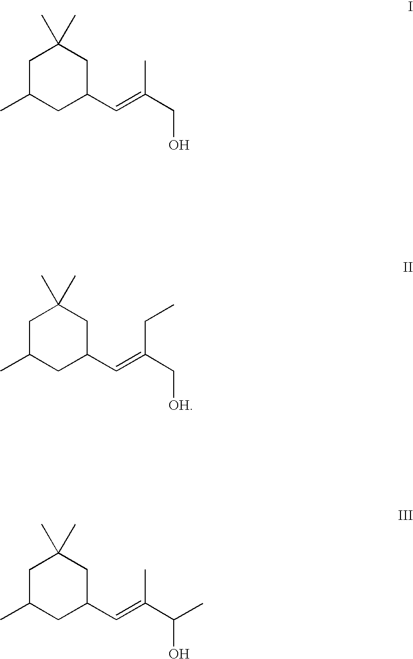

2. The method of claim 1 where in the fragrance is incorporated into a product selected from perfumes, colognes, cosmetic products, personal care products and air fresheners.

3. The method of claim 2 where in the level is from about 0.1% to about 3% by weight.

4. A compound having the structure (I) set forth in claim 1: 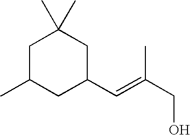
2-Methyl-3(3,5,5-trimethyl cyclo hexane)-2-propenol (I):

5. A compound having the structure (II) set forth in claim 1: 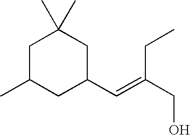
2-Ethyl-3(3,5,5-trimethyl cyclo hexane)-2-propenol (II)

6. A compound having the structure (III) set forth in claim 1: 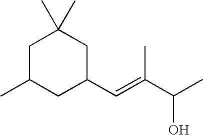
2-Methyl-4(3,5,5-trimethyl cyclo hexane)-3-propen-2-ol (III)

7. A perfume composition incorporating 2-Methyl-3(3,5,5-trimethyl cyclo hexane)-2-propenol (I):

8. A perfume composition incorporating 2-Ethyl-3(3,5,5-trimethyl cyclo hexane)-2-propenol (II)

9. A perfume composition incorporating 2-Methyl-4(3,5,5-trimethyl cyclo hexane)-3-propen-2-ol (III)

10. A method of synthesis of compounds I, II & III claimed in claim 1 consisting of reacting 3,5,5-trimethyl cyclohexylcarboxaldehyde with propinaldehyde, butyraldehyde or methylethyl ketone respectively followed by reduction.
